(19) United States
(12) Patent Application Publication
(10) Pub. No.: US 2017/0113992 A1
(43) Pub. Date: Apr. 27, 2017

(54) METHODS AND MATERIALS FOR HYDROLYZING POLYESTERS

(71) Applicant: East China University of Science and Technology, Shanghai (CN)

(72) Inventors: Guiping CAO, Shanghai (CN); Xuekun LI, Shanghai (CN); Kaiyue HAN, Shanghai (CN); Chen MENG, Shanghai (CN)

(73) Assignee: EAST CHINA UNIVERSITY OF SCIENCE AND TECHNOLOGY, Shanghai (CN)

(21) Appl. No.: 15/301,715

(22) PCT Filed: Apr. 2, 2014
(86) PCT No.: PCT/CN2014/000366

§ 371 Date: Oct. 3, 2016

(51) Int. Cl. C07C 51/09; C07C 29/80; C07C 51/44; B01J 27/053; B01J 23/22; B01J 37/02; B01J 21/06; B01J 23/26; B01J 23/10; B01J 21/04; B01J 23/30; B01J 21/12; C07C 29/09; B01J 23/14

(52) U.S. Cl. CPC C07C 51/09; C07C 29/095; C07C 29/80; C07C 51/44; B01J 27/053; B01J 23/22; B01J 23/14; B01J 21/063; B01J 23/26; B01J 21/066; B01J 23/10; B01J 21/04; B01J 23/30; B01J 21/12; B01J 37/0201

(57) ABSTRACT

The present application relates to methods of hydrolyzing a polyester by contacting the polyester with a solid acid catalyst. Also disclosed are solid acid catalysts that are useful for hydrolyzing a polyester and methods of making the solid acid catalysts. Furthermore, compositions including one or both of a dicarboxylic acid and a diol, and at least one solid acid catalyst are also disclosed.

### BACKGROUND

A copolyester of polyprotic acid and polyol, such as PET (polyethylene terephthalate) and PBT (polybutylene terephthalate), is a thermoplastic semicrystalline polymer with a wide range of applications. Taking PET for example, it has a glass transition temperature of 80° C., a melting temperature of 250-255° C., and a decomposition temperature of 353° C. Its structural formula is as follows:

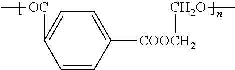

At present, a commonly used method for producing PET and PBT is through esterification and condensation polymerization between terephthalic acid (TPA) and ethylene glycol (EG) or butanediol, wherein the monomer terephthalic acid is obtained from the oxidation of p-xylene (PX), ethylene glycol is obtained from the oxidation of ethylene, and butanediol is obtained through a biological method or from the oxidation of butadiene. The production of the monomers can involve long routes of synthesis, high costs and severe pollution.

Currently, the two main industrial methods for the chemical recovery of PET are hydrolysis and alcoholysis. Alcoholysis is divided into methanolysis and glycol alcoholysis. PET can be depolymerized in methanol at a high temperature and under a high pressure, and the products are dimethyl terephthalate (DMT) and EG. Glycol alcoholysis is another chemical recovery method for PET depolymerization, and the reaction products are bis(2-hydroxyethyl) terephthalate (BHET) and EG. BHET has a wide range of applications in the synthesis of unsaturated resins and polyurethanes. However, the alcoholysis method has its disadvantages including high cost due to the separation and purification of ethylene glycol, methanol and phthalic acid derivatives from the reaction products; loss of efficacy of the catalyst caused by the presence of water in the reaction process; and more complexity in the process and higher operation requirement relative to the hydrolysis method.

Hydrolysis is mainly divided into three types: basic hydrolysis, neutral hydrolysis and acidic hydrolysis. Basic hydrolysis is typically conducted in aqueous KOH or NaOH solution at a certain concentration, and the products are EG and terephthalate. EG can be recovered through evaporation when the products are heated to more than 300° C. This process requires a lot of energy and the remaining solution has to be neutralized by a strong acid to obtain pure TPA. This can result in the production of a lot of inorganic salts and waste water. Although basic hydrolysis is simpler and cheaper than alcoholysis process, the waste liquid after the reaction can easily pollute the environment. In addition, traditional basic hydrolysis reaction requires a higher temperature and a longer reaction time.

Generally, neutral hydrolysis is carried out in water vapor, and the products after the hydrolysis are TPA and EG. Since the neutral hydrolysis method will not produce difficult-to-handle inorganic salts, it will not result in corrosion of equipment by a concentrated acid or concentrated alkaline, and is environment-friendly. The disadvantage of the method is that all the impurities in PET remains in TPA, hence the purity of the reaction product is lower than that of acidic hydrolysis or basic hydrolysis. Therefore, the neutral hydrolysis method can be quite a complex purification process, thereby increasing the recovery cost.

In comparing the above-described methods for degrading PET, the biggest advantage of traditional acidic hydrolysis lies in lower reaction temperature. The reaction can be carried out at a temperature lower than 100° C. However, concentrated sulfuric acid, nitric acid, phosphoric acid or other strong inorganic acids are most commonly used in the hydrolysis process. As to the degradation of PET in these inorganic acid solutions, there are still problems such as high production cost due to the recovery of a great amount of concentrated sulfuric acid and the purification of EG from the sulphuric acid after the acidic hydrolysis; production of a lot of inorganic salts and waste water; and relatively serious corrosion of the reaction system by the concentrated acid. Thus, it will be desirable to provide methods of hydrolyzing polyesters that at least ameliorate or overcome the disadvantages described above.

### SUMMARY

The foregoing summary is illustrative only and is not intended to be in any way limiting. In addition to the illustrative aspects, embodiments, and features described above, further aspects, embodiments, and features will become apparent by reference to the drawings and the following detailed description.

Some embodiments disclosed herein include a method of hydrolyzing a polyester, the method includes providing a first mixture comprising at least one polyester, and at least one solid acid catalyst; and contacting the first mixture with carbon dioxide under conditions sufficient to hydrolyze the at least one polyester to form a second mixture comprising at least one dicarboxylic acid and at least one diol.

Some embodiments disclosed herein include a solid acid catalyst including: at least one metal oxide and at least one strong acidic ion.

Some embodiments disclosed herein include a method of making a solid acid catalyst, the method includes contacting at least one metal oxide with at least one strong acid ion to form a mixture comprising the solid acid catalyst.

Some embodiments disclosed herein include a composition including: at least one polyester, and at least one solid acid catalyst configured to hydrolyze the polyester to form at least one dicarboxylic acid and at least one diol, the solid acid catalyst comprising at least one metal oxide and at least one strong acidic ion.

Some embodiments disclosed herein include a composition including: one or both of a dicarboxylic acid and a diol; and at least one solid acid catalyst configured to hydrolyze a polyester to the one or both of the dicarboxylic acid and the diol, the solid acid catalyst comprising at least one metal oxide and at least one strong acidic ion.

### DETAILED DESCRIPTION

The disclosed embodiments provide methods of hydrolyzing a polyester by contacting the polyester with a solid acid catalyst under a supercritical CO₂ environment. The methods according to the disclosed embodiments generally do not involve energy-intensive reaction conditions and can be carried out at low reaction temperatures and pressures. The methods according to the disclosed embodiments generally involve simple reaction processes, for example, a polyester hydrolysis reaction catalyzed by a solid acid under supercritical CO₂ environment. Energy consumption of the hydrolysis reaction can accordingly be low. The solid acid catalyst can also be recovered after the reaction and can be recycled.

In some embodiments, the method of hydrolyzing a polyester includes providing a first mixture having at least one polyester, and at least one solid acid catalyst; and contacting the first mixture with carbon dioxide under conditions sufficient to hydrolyze the at least one polyester to form a second mixture having at least one dicarboxylic acid and at least one diol.

The polyester may be represented by Formula (I):

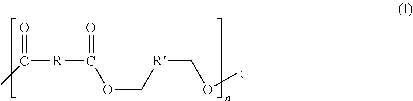

wherein n is 100-500, R is absent, C₁₋₈ alkylene, phenylene, naphthylene, or

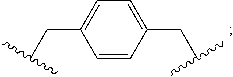 [SMILES: *Cc1ccc(C*)cc1]

and R′ is absent, C₁₋₆ alkylene or C₃₋₈ cycloalkylene. R and R′ can each be optionally substituted. In some embodiments, the polyester is selected from polyethylene terephthalate, polybutylene terephthalate, polybutylene succinate, polyhexylene sebacate, polybutylene naphthalate, polycyclohexylene dimethylene terephthalate, polyethylene naphthalate, polypropylene adipate, polyethylene glycol malonate, polyethyleneglycol glutarate, poly (tetraethylene glycol suberate) (PTEGSub), poly[di(ethylene glycol) adipate], poly(ethylene adipate), poly(propylene adipate), poly(butylene adipate), poly(glutarate adipate), poly(hexamethylene adipate), poly(octyldiester adipate), poly(ethylene succinate), poly(trimethylene succinate), poly (butylsuccinate diesters), poly(hexamethylene succinate), poly(octyl succinate diester), poly(ethylene sebacate), poly(propylene sebacate esters), poly(butylene sebacate), poly(hexamethylene sebacate), poly(octylsebacate diesters), and any combination thereof. In some embodiments, the polyester is in a block form, a granular form, a powder form, or any combination thereof.

In some embodiments, the solid acid catalyst may include at least one metal oxide and at least one strong acidic ion. The metal oxide (MₓO_{y}) may be Fe₂O₃, Fe₃O₄, TiO₂, Al₂O₃, ZrO₂, V₂O₅, WO₃, Cr₂O₃, CeO₂, SnO₂, SiO₂—Al₂O₃, ZrO₂—WO₃, ZrO₂—Al₂O₃, TiO₂—Al₂O₃, ZrO₂—Al₂O₃—WO₃, SiO₂—V₂O₅, SiO₂—TiO₂, Al₂O₃—Cr₂O₃, or any combination thereof. In some embodiments, the metal oxide is in hydrated form. The strong acidic ion may be SO₄²⁻, NO₃⁻, PO₄³⁻, or any combination thereof. In some embodiments, the solid acid catalyst may be SO₄²⁻/MₓO_{y}, NO₃⁻/MₓO_{y}, or PO₄³⁻/MₓO_{y}. Examples of solid acid catalysts include SO₄²⁻/V₂O₅, SO₄²⁻/SnO₂, SO₄²⁻/TiO₂—Al₂O₃, SO₄²⁻/Al₂O₃—Cr₂O₃, SO₄²⁻/ZrO₂, SO₄²⁻/CeO₂, SO₄²⁻/ZrO₂—Al₂O₃, SO₄²⁻/Al₂O₃, SO₄²⁻/Cr₂O₃, SO₄²⁻/ZrO₂—WO₃, SO₄²⁻/SiO₂—V₂O₅, SO₄²⁻/TiO₂, SO₄²⁻/WO₃, SO₄²⁻/ZrO₂—Al₂O₃—WO₃, SO₄²⁻/SiO₂—Al₂O₃, NO₃⁻/SiO₂—Al₂O₃, and PO₄³⁻/SiO₂—Al₂O₃.

In some embodiments, the first mixture may further include a liquid medium. The liquid medium may be water, including deionized water, distilled water or tap-water.

In some embodiments, contacting the first mixture with the carbon dioxide occurs at an elevated temperature, such as a temperature of about 50° C. to about 350° C., about 100° C. to about 250° C., or about 150° C. to about 200° C. For example, the first mixture may be contacted with the carbon dioxide at a temperature of about 50° C., about 100° C., about 150° C., about 200° C., about 250° C., about 300° C., about 350° C., or a temperature between any of these values. In some embodiments, the carbon dioxide is supercritical carbon dioxide. In some embodiments, contacting the first mixture with the carbon dioxide occurs at an elevated pressure, such as a pressure of about 7.5 MPa to about 25.5 MPa, about 8 MPa to about 20 MPa, or about 9 MPa to about 15 MPa. For example, the first mixture may be contacted with the carbon dioxide at a pressure of about 7.5 MPa, about 10 MPa, about 12.5 MPa, about 15.0 MPa, about 17.5 MPa, about 20 MPa, about 22.5 MPa, about 25.5 MPa or a pressure between any of these values. In some embodiments, contacting the first mixture with the carbon dioxide occurs for a period of time, such as about 0.1 hour to about 96 hours, about 1 hour to about 72 hours, or about 3 hour to about 24 hours. For example, contacting the first mixture with the carbon dioxide may occur for about 0.1 hour, about 10 hours, about 20, hours, about 30 hours, about 40 hours, about 50 hours, about 60 hours, about 70 hours, about 80 hours, about 90 hours, about 96 hours or a time period between any of these values.

In some embodiments, the method may further include recovering the dicarboxylic acid and the diol from the second mixture. The recovering of the dicarboxylic acid and the diol from the second mixture may include separating the second mixture into a first solid phase and a first liquid phase, and separating the diol from the first liquid phase. In some embodiments, separating the second mixture into the first solid phase and the first liquid phase may include filtering the second mixture. In some embodiments, separating the diol from the first liquid phase may include distilling the first liquid phase. In some embodiments, recovering the dicarboxylic acid and the diol from the second mixture may include separating the second mixture into a first solid phase and a first liquid phase, contacting the first solid phase with a solvent or an alkali to form a second solid phase and a second liquid phase; separating the dicarboxylic acid from the second liquid phase. The separating of the second mixture into the first solid phase and the first liquid phase may include filtering the second mixture. The separating of the dicarboxylic acid from the second liquid phase may include distilling the second liquid phase. In some embodiments, the solvent is an organic solvent selected from chloroform, ethanol, ethylether, dimethylformamide (DMF), diethylformamide (DEF), and dimethyl sulfoxide (DMSO). In some embodiments, the alkali is selected from sodium hydroxide and potassium hydroxide.

In some embodiments, the method may further include recovering the solid acid catalyst from the second mixture. The recovering of the solid acid catalyst from the second mixture may include separating the second mixture into a first solid phase and a first liquid phase, contacting the first solid phase with a solvent or an alkali to form a second solid phase and a second liquid phase, and washing the second solid phase to obtain the solid acid catalyst. The separating of the second mixture into the first solid phase and the first liquid phase may include filtering the second mixture. In some embodiments, the solvent is an organic solvent selected from chloroform, ethanol, ethylether, dimethylformamide (DMF), diethylformamide (DEF), and dimethyl sulfoxide (DMSO). In some embodiments, the alkali is selected from sodium hydroxide and potassium hydroxide.

In some embodiments, the method may further include converting a salt of the dicarboxylic acid to the dicarboxylic acid by contacting the second liquid phase with an acid. The dicarboxylic acid may then be separated from the second liquid phase. In some embodiments, the acid is an inorganic acid selected from hydrochloric acid, sulfuric acid, nitric acid, and phosphoric acid. In some embodiments, the dicarboxylic acid can be separated from the second liquid phase by distilling the second liquid phase. The dicarboxylic acid may be represented by R(COOH)₂, wherein R is as defined above. In some embodiments, R is ethylene, butylene, octylene, phenylene, or naphthylene. Examples of the dicarboxylic acid include terephthalic acid, sebacic acid, p-naphthalic acid, 2,6-naphthalic acid, hexanedioic acid, succinic acid, propanedioic acid, azelaic acid, pimelic acid, suberic acid, glutaric acid, or any combination thereof.

The diol can be separated from the first liquid phase. In some embodiments, the first liquid phase is distilled to separate and recover the diol. In some embodiments, the diol is R′(CH₂OH)₂, wherein R′ is as defined above. In some embodiments, R′ is cyclohexylene or —(CH₂)ₓ—, wherein x is 0, 1, 2, 3, or 4. Examples of diols include ethylene glycol, propylene glycol, butanediol, hexanediol, cyclohexanedimethanol, heptanediol, octanediol, or any combination thereof.

Some embodiments provide a solid catalyst that includes at least one metal oxide and at least one strong acidic ion. The metal oxide and the acidic ion can for example be those as described above. Some embodiments provide a method of making a solid acid catalyst. The solid acid catalyst can be formed by contacting at least one metal oxide with at least one strong acid ion (for example, a strong acid, a salt of the strong acid, or both) to form a mixture that includes the solid acid catalyst. The contacting of the at least one metal oxide with the at least one strong acid ion may for example be performed by stirring, and can occur for a period of time, such as about 0.5 hours to about 72 hours, about 6 hours to about 60 hours, about 12 hours to about 48 hours, or about 24 hours to about 36 hours. For example, the contacting of the at least one metal oxide with the at least one strong acid ion may occur for about 6 hours, about 12 hours, about 18 hours, about 24 hours, about 30 hours, about 36 hours, about 42 hours, about 48 hours, about 54 hours, about 60 hours, about 66 hours, about 72 hours, or a time period between any of these values. The at least one strong acid ion can have generally any concentration, such as a concentration of about 0.05 mol/L to about 5 mol/L, about 0.1 mol/L to about 3 mol/L, or about 0.5 mol/L to about 1 mol/L in the mixture. For example, the at least one strong acid ion can have a concentration of about 0.05 mol/L, about 0.1 mol/L, about 0.2 mol/L, about 0.5 mol/L, about 0.8 mol/L, about 1 mol/L, about 1.5 mol/L, about 2 mol/L, about 2.5 mol/L, about 3 mol/L, about 3.5 mol/L, about 4 mol/L, about 4.5 mot/L, about 5 mol/L, or a concentration between any of these valves. In some embodiments, the method of making the solid catalyst may further include isolating the solid catalyst from the mixture, and drying the solid acid catalyst. The isolating of the solid catalyst from the mixture can for example be performed by filtering the mixture.

The isolated solid catalyst can be dried by heating the solid acid catalyst at a first temperature and then at a higher second temperature. For example, the first temperature can be about 100° C. to about 150° C., or about 110° C., and the second temperature can be about 400° C. to about 600° C., or about 500° C. to about 600° C. In some embodiments, the solid acid catalyst is heated at the first temperature for a period of time, such as about 6 hours to about 50 hours, then at the second temperature for a period of time, such as about 1 hour to about 12 hours or about 4 hours to about 7 hours. In some embodiments, the solid acid catalyst can be heated at the first temperature for about 12 hours to about 48 hours, or about 24 hours to about 36 hours, and then at the second temperature for about 3 hours to about 9 hours, about 5 hours to about 7 hours, about 4.5 hours to about 6.5 hours, or about 5 hours to about 6 hours.

Some embodiments provide a composition including at least one polyester and at least one solid acid catalyst. The at least one solid acid catalyst can be configured to hydrolyze the polyester to form at least a dicarboxylic acid and a diol. The at least one solid acid catalyst may include at least one metal oxide and at least one strong acidic ion.

Some embodiments provide a composition including one or both of a dicarboxylic acid and a diol and at least one solid acid catalyst. The solid acid catalyst can be configured to hydrolyze a polyester to the one or both of the dicarboxylic acid and the diol, the solid acid catalyst including at least one metal oxide and at least one strong acidic ion. In some embodiments, the composition may also include at least one partially hydrolyzed polyester.

### Examples

The present invention is further illustrated by the following examples. However, the scope of the present invention is not limited to these examples.

In each of the following examples, the volume of the sealable reactor used is 500 ml, the mass of polyester material was 50 g, the amount of solid acid catalyst was 10 g, and the mass of water used in the reaction was 100 g. The volume of the mixture of polyester material, solid acid catalyst and water accounted for about ⅓ of the volume of the reactor. The stirring rate was 100 rpm during the reaction. After the reaction was completed, the venting rate of the reactor was 0.5 MPa/min.

In the following examples, the polyesters used in Examples 1, 4, 7, 10, 13, 16, 17, 18 and 19 were powder materials with a particle size of 0.1-1 mm; the polyesters used in Examples 2, 5, 8, 11 and 14 were granular materials with a particle size of 1-3 mm, or cuboids with a size of 1-3 mm in length, 1-3 mm in width and 0.5-1.5 mm in height; and the polyesters used in Examples 3, 6, 9, 12 and 15 were block materials which were cuboids with a size of 3-10 mm in length, 3-10 mm in width and 1.5-3 mm in height.

In the following examples, the degree of hydrolysis of the polyesters and the yields of dicarboxylic acid and polyol are both obtained by weighing method (for example, mass percent). For example, when the polyester is PET, the calculation method is as follows:

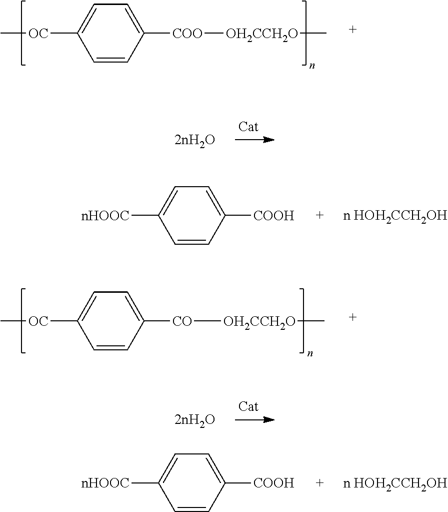

$$\begin{matrix}n ( mol ) \times 192 ( g \text{/} mol ) & n ( mol ) \times 166 ( g \text{/} mol ) & n ( mol ) \times 62 ( g \text{/} mol ) \\ {m}_{0 , PET} ( g ) & \frac{166}{192} {m}_{0 , PET} ( g ) & \frac{62}{192} {m}_{0 , PET} ( g )\end{matrix}$$

Degree of hydrolysis of PET:

$${d}_{h} ( degradation of PET ) = ( 1 - \frac{{m}_{Res , PET}}{{m}_{0 , PET}} ) \times 100 \%$$

Yield of TPA:

$${c}_{TPA} ( yield of TPA ) = \frac{{m}_{TPA}}{\frac{166}{192} {m}_{0 , PET}} \times 100 \%$$

Yield of EG:

$${c}_{EG} ( yield of EG ) = \frac{{m}_{EG}}{\frac{62}{192} {m}_{0 , PET}} \times 100 \%$$

TABLE 1
A list of parameters used in Examples 1 to 19

| | | | Conc. of | | | | |
|---|---|---|---|---|---|---|---|
| | | | acid | Stirring and | Reaction | CO₂ | |
| | | | radical, | impregnating | temp., | pressure, | Reaction |
| No. | Polyester | Solid Acid Catalyst | mol/L | time, h | ° C. | MPa | time, h |
| 1 | PET | SO₄²⁻/V₂O₅ | 1.7 | 41.2 | 230 | 19.5 | 60.0 |
| 2 | PBT | SO₄²⁻/SnO₂ | 3.7 | 0.5 | 90 | 12.3 | 36.0 |
| 3 | PET | SO₄²⁻/TiO₂—Al₂O₃ | 0.05 | 45.6 | 290 | 25.5 | 18.0 |
| 4 | PHS | SO₄²⁻/Al₂O₃—Cr₂O₃ | 2.0 | 3.0 | 150 | 18.3 | 3.0 |
| 5 | PET | SO₄²⁻/ZrO₂ | 4.0 | 50.0 | 350 | 11.1 | 0.5 |
| 6 | PBN | SO₄²⁻/CeO₂ | 0.3 | 9.0 | 210 | 24.3 | 96.0 |
| 7 | PET | SO₄²⁻/ZrO₂—Al₂O₃ | 2.4 | 54.4 | 70 | 17.1 | 50.0 |
| 8 | PCT | SO₄²⁻/Al₂O₃—Cr₂O₃ | 4.3 | 18.0 | 270 | 9.9 | 30.0 |
| 9 | PET | SO₄²⁻/Al₂O₃ | 0.7 | 58.8 | 130 | 23.1 | 12.0 |
| 10 | PEN | SO₄²⁻/Cr₂O₃ | 2.7 | 23.6 | 330 | 15.9 | 2.0 |
| 11 | PET | SO₄²⁻/ZrO₂—WO₃ | 4.7 | 63.2 | 190 | 8.7 | 0.1 |
| 12 | PPA | SO₄²⁻/SiO₂—V₂O₅ | 1 | 28.0 | 50 | 21.9 | 72.0 |
| 13 | PET | SO₄²⁻/TiO₂ | 3 | 67.6 | 250 | 14.7 | 50.0 |
| 14 | PBT | SO₄²⁻/WO₃ | 5 | 32.4 | 110 | 7.5 | 24.0 |
| 15 | PBS | SO₄²⁻/ZrO₂—Al₂O₃—WO₃ | 1.4 | 72.0 | 310 | 20.7 | 6.0 |
| 16 | PET | SO₄²⁻/SiO₂—Al₂O₃ | 3.4 | 36.8 | 170 | 13.5 | 1.0 |
| 17 | PET | NO₃⁻/SiO₂—Al₂O₃ | 3.4 | 36.8 | 170 | 13.5 | 1.0 |
| 18 | PET | PO₄³⁻/SiO₂—Al₂O₃ | 3.4 | 36.8 | 170 | 13.5 | 1.0 |
| 19 | PET | TiO₂—Al₂O₃ | — | — | 290 | 25.5 | 18.0 |

### Example 1: Hydrolysis of PET with SO₄²⁻/V₂O₅

50 g of V(NO₃)₅ was dissolved in 1 L of distilled water. The solution was stirred quickly and aqueous ammonia having a concentration of 25% by weight in water was added dropwise until the pH of the solution reached 9. At this pH, a precipitate of V₂O₅.xH₂O solid was formed in the solution. The solution was left to stand for 24 hours, followed by suction filtering to obtain the precipitate. The precipitate from the solution was dried at 110° C. for 12 hour. The obtained V₂O₅.xH₂O solid was pulverized to smaller than 100 mesh (0.15 mm) and added to 1.7 mol/L H₂SO₄ solution. The amount of H₂SO₄ solution was 25 mL solution/g V₂O₅.xH₂O solid. The mixture of V₂O₅.xH₂O solid and the H₂SO₄ solution was stirred for 41.2 hours and filtered. After filtration, the resulting SO₄²⁻/V₂O₅.xH₂O was dried at 110° C. for 12 hours, and calcinated at 550° C. for 6 hours to obtain pulverous SO₄²⁻/V₂O₅, an ultra-strong solid acid catalyst, which was weighed and recorded.

50 g of powder PET with a particle size of 0.1 mm, 10 g of SO₄²⁻/V₂O₅ catalyst and 100 g of deionized water were charged into a reactor. The reactor was sealed, stirred and heated to a constant temperature of 230° C. CO₂ was then charged, resulting in a reactor pressure of 19.5 MPa, and the reaction was carried out for 60 hours. After the reaction was completed, the pressure within the reactor was reduced to atmospheric pressure at a venting rate of 0.5 MPa/min. The products in the reactor were filtered and then the liquid phase was removed and subjected to distillation operation. After the remnant water was evaporated, pure ethylene glycol was obtained and then weighed to calculate the yield. The solid phase was dried, dissolved in the corresponding solvent for 6 hours while stirring, and then filtered again. The liquid phase was removed for distillation. After the solvent was evaporated, pure terephthalic acid was obtained and weighed to calculate the yield. The solid phase was rinsed with deionized water to remove redundant solvent. After filtration, the residual solid was dried to constant weight to obtain SO₄²⁻/V₂O₅ catalyst and residual PET, which were weighed to obtain the degree of hydrolysis of PET.

### Example 2: Hydrolysis of PBT with SO₄²⁻/SnO₂

50 g of SnCl₄.5H₂O was dissolved in 1 L of distilled water. The solution was stirred quickly and aqueous ammonia having a concentration of 25% by weight in water was added dropwise until the pH of the solution reached 9. At this pH, a precipitate of SnO₂.xH₂O solid was formed in the solution. The solution was left to stand for 24 hours, followed by suction filtering to obtain the precipitate. The precipitate from the solution was dried at 110° C. for 12 hours. The obtained SnO₂.xH₂O solid was pulverized to smaller than 100 mesh (0.15 mm) and added to 3.7 mol/L Sn(SO₄)₂ solution. The amount of H₂SO₄ solution was 25 mL solution/g SnO₂.xH₂O solid. The mixture of SnO₂.xH₂O solid and the H₂SO₄ solution was stirred for 0.5 hours and filtered. After filtration, the resulting SO₄²⁻/SnO₂.xH₂O was dried at 110° C. for 12 hours, and calcinated at 500° C. for 6 hours to obtain pulverous SO₄²⁻/SnO₂, an ultra-strong solid acid catalyst, which was weighed and recorded.

50 g of granular PBT with a particle size of 1 mm, 10 g of SO₄²⁻/SnO₂ catalyst and 100 g of deionized water were charged into a reactor. The reactor was sealed, stirred and heated to a constant temperature of 90° C. CO₂ was then charged, resulting in a reactor pressure of 12.3 MPa, and the reaction was carried out for 36 hours. After the reaction was completed, the pressure within the reactor was reduced to atmospheric pressure at a venting rate of 0.5 MPa/min. The products in the reactor were filtered and then the liquid phase was removed and subjected to distillation operation. After water was evaporated, pure butanediol was obtained and then weighed to calculate the yield. The solid phase was dried, dissolved in a special organic solvent for 6 hours while stirring, and then filtered again. The liquid phase was removed for distillation. After the solvent was evaporated, pure terephthalic acid was obtained and weighed to calculate the yield. The solid phase was rinsed with deionized water to remove redundant solvent. After filtration, the residual solid was dried to constant weight to obtain SO₄²⁻/SnO₂ catalyst and residual PBT, which were weighed to obtain the degree of hydrolysis of PBT.

### Example 3: Hydrolysis of PET with SO₄²⁻/TiO₂—Al₂O₃

30 g of Ti(SO₄)₂.8H₂O and 20 g of Al(NO₃)₃.9H₂O were dissolved in 1 L of distilled water. The solution was stirred quickly and aqueous ammonia having a concentration of 25% by weight in water was added dropwise until the pH of the solution reached 9. At this pH, a precipitate of TiO₂—Al₂O₃.xH₂O solid was formed in the solution. The solution was left to stand for 24 hours, followed by suction filtering to obtain the precipitate. The precipitate from the solution was dried at 110° C. for 12 hours. The obtained TiO₂—Al₂O₃.xH₂O solid was pulverized to smaller than 100 mesh (0.15 mm) and added to 0.05 mol/L H₂SO₄ solution. The amount of H₂SO₄ solution was 25 mL solution/g TiO₂—Al₂O₃.xH₂O solid. The mixture of TiO₂—Al₂O₃.xH₂O solid and the H₂SO₄ solution was stirred 45.6 and filtered. After filtration, the resulting SO₄²⁻/TiO₂—Al₂O₃.xH₂O was dried at 110° C. for 12 h, and calcinated at 500° C. for 6 hours to obtain pulverous SO₄²⁻/TiO₂—Al₂O₃, a solid acid catalyst, which was weighed and recorded.

50 g of block PET with a length of 3 mm, a width of 3 mm and a height of 1.5 mm, 10 g of SO₄²⁻/TiO₂—Al₂O₃ catalyst and 100 g of deionized water were charged into a reactor. The reactor was sealed, stirred and heated to a constant temperature of 290° C. CO₂ was then charged, resulting in a reactor pressure of 25.5 MPa, and the reaction was carried out for 18 hours. After the reaction was completed, the pressure within the reactor was reduced to atmospheric pressure at a venting rate of 0.5 MPa/min. The products in the reactor were filtered and then the liquid phase was removed and subjected to distillation operation. After water was evaporated, pure ethylene glycol was obtained and then weighed to calculate the yield. The solid phase was dried, dissolved in a special organic solvent for 6 hours while stirring, and then filtered again. The liquid phase was removed for distillation. After the solvent was evaporated, pure terephthalic acid was obtained and weighed to calculate the yield. The solid phase was rinsed with deionized water to remove redundant solvent. After filtration, the residual solid was dried to constant weight to obtain SO₄²⁻/TiO₂—Al₂O₃ catalyst and residual PET, which were weighed to obtain the degree of hydrolysis of PET.

### Example 4: Hydrolysis of PHS with SO₄²⁻/Al₂O₃—Cr₂O₃

30 g of Cr(NO₃)₃.9H₂O and 20 g of Al(NO₃)₃.9H₂O were dissolved in 1 L of distilled water. The solution was stirred quickly and aqueous ammonia having a concentration of 25% by weight in water was added dropwise until the pH of the solution reached 9. At this pH, a precipitate of Al₂O₃—Cr₂O₃.xH₂O solid was formed in the solution. The solution was left to stand for 24 hours, followed by suction filtering to obtain the precipitate. The precipitate from the solution was dried at 110° C. for 12 hours. The obtained Al₂O₃—Cr₂O₃.xH₂O solid was pulverized to smaller than 100 mesh (0.15 mm) and added to 2.0 mol/L Cr₂(SO₄)₃ solution. The amount of H₂SO₄ solution was 25 mL solution/g Al₂O₃—Cr₂O₃.xH₂O solid. The mixture of Al₂O₃—Cr₂O₃.xH₂O solid and the H₂SO₄ solution was stirred 3 hours and filtered. After filtration, the resulting SO₄²⁻/Al₂O₃—Cr₂O₃.xH₂O was dried at 110° C. for 12 hours, and calcinated at 500° C. for 6 hours to obtain pulverous SO₄²⁻/Al₂O₃—Cr₂O₃, a solid acid catalyst, which was weighed and recorded.

50 g of powder PHS with a particle size of 0.5 mm, 10 g of SO₄²⁻/Al₂O₃—Cr₂O₃ catalyst and 100 g of deionized water were charged into a reactor. The reactor was sealed, stirred and heated to a constant temperature of 150° C. CO₂ was then charged, resulting in a reactor pressure of 18.3 MPa, and the reaction was carried out for 3 hours. After the reaction was completed, the pressure within the reactor was reduced to atmospheric pressure at a venting rate of 0.5 MPa/min. The products in the reactor were filtered and then the liquid phase was removed and subjected to distillation operation. After water and hexanediol were evaporated and collected at different distillation ranges by controlling the temperature, pure hexanediol and sebacic acid crystals were obtained, respectively, and then weighed to calculate the yield. The residual solid was dried to constant weight to obtain SO₄²⁻/TiO₂—Al₂O₃ catalyst and residual PHS, which were weighed to obtain the degree of hydrolysis of PHS.

### Example 5: Hydrolysis of PET with SO₄²⁻/ZrO₂

50 g of ZrO(NO₃)₂.2H₂O was dissolved in 1 L of distilled water. The solution was stirred quickly and aqueous ammonia having a concentration of 25% by weight in water was added dropwise until the pH of the solution reached 9. At this pH, a precipitate of ZrO₂.xH₂O solid was formed in the solution. The solution was left to stand for 24 hours, followed by suction filtering to obtain the precipitate. The precipitate in the solution was dried at 110° C. for 12 hours. The obtained ZrO₂.xH₂O solid was pulverized to smaller than 100 mesh (0.15 mm) and added to 4.0 mol/L ZrSO₄ solution. The amount of H₂SO₄ solution was 25 mL solution/g ZrO₂.xH₂O solid. The mixture of ZrO₂.xH₂O solid and the H₂SO₄ solution was stirred 50 hours and filtered. After filtration, the resulting SO₄²⁻/ZrO₂.xH₂O was dried at 110° C. for 12 h, and calcinated at 550° C. for 6 hours to obtain pulverous SO₄²⁻/ZrO₂, an ultrastrong solid acid catalyst.

50 g of granular PET with a particle size of 1.5 mm, 10 g of SO₄²⁻/ZrO₂ catalyst and 100 g of deionized water were charged into a reactor. The reactor was sealed, stirred and heated to a constant temperature of 350° C. CO₂ was then charged, resulting in a reactor pressure of 11.1 MPa, and the reaction was carried out for 0.5 hours. After the reaction was completed, the pressure within the reactor was reduced to atmospheric pressure at a venting rate of 0.5 MPa/min. The products in the reactor were filtered and then the liquid phase was removed and subjected to distillation operation. After water was evaporated, pure ethylene glycol was obtained and then weighed to calculate the yield. The solid phase was dried, dissolved in a special organic solvent for 6 hours while stirring, and then filtered again. The liquid phase was removed for distillation. After the solvent was evaporated, pure terephthalic acid was obtained and weighed to calculate the yield. The solid phase was rinsed with deionized water to remove redundant solvent. After filtration, the residual solid was dried to constant weight to obtain SO₄²⁻/ZrO₂ catalyst and residual PET, which were weighed to obtain the degree of hydrolysis of PET.

### Example 6: Hydrolysis of PBN with SO₄²⁻/CeO₂

50 g of Ce(NO₃)₃.6H₂O was dissolved in 1 L of distilled water. The solution was stirred quickly and aqueous ammonia having a concentration of 25% by weight in water was added dropwise until the pH of the solution reached 9. At this pH, a precipitate of CeO₂.xH₂O solid was formed in the solution. The solution was left to stand for 24 hours, followed by suction filtering to obtain the precipitate. The precipitate in the solution was dried at 110° C. for 12 hours. The obtained CeO₂.xH₂O solid was pulverized to smaller than 100 mesh (0.15 mm) and added to 0.3 mol/L Ce₂(SO₄)₃ solution. The amount of H₂SO₄ solution was 25 mL solution/g CeO₂.xH₂O solid. The mixture of CeO₂.xH₂O solid and the H₂SO₄ solution was stirred for 9 hours and filtered. After filtration, the resulting SO₄²⁻/CeO₂.xH₂O was dried at 110° C. for 12 hours, and calcinated at 550° C. for 6 hours to obtain pulverous SO₄²⁻/CeO₂, an ultrastrong solid acid catalyst.

50 g of block PBN with a length of 5 mm, a width of 5 mm and a height of 2 mm, 10 g of SO₄²⁻/CeO₂ catalyst and 100 g of deionized water were charged into a reactor. The reactor was sealed, stirred and heated to a constant temperature of 210° C. CO₂ was then charged, resulting in a reactor pressure of 24.3 MPa, and the reaction was carried out for 96 hours. After the reaction was completed, the pressure within the reactor was reduced to atmospheric pressure at a venting rate of 0.5 MPa/min. The products in the reactor were filtered and then the liquid phase was removed and subjected to distillation operation. After water was evaporated, pure butanol was obtained and then weighed to calculate the yield. The solid phase was dried, dissolved in a special organic solvent for 6 hours while stirring, and then filtered again. The liquid phase was removed for distillation. After the solvent was evaporated, pure p-naphthalic acid was obtained and weighed to calculate the yield. The solid phase was rinsed with deionized water to remove redundant solvent. After filtration, the residual solid was dried to constant weight to obtain SO₄²⁻/CeO₂ catalyst and residual PBN, which were weighed to obtain the degree of hydrolysis of PBN.

### Example 7: Hydrolysis of PET with SO₄²⁻/Al₂O₃—Cr₂O₃

30 g of Zr(NO₃)₃.5H₂O and 20 g of Al(NO₃)₃.9H₂O were dissolved in 1 L of distilled water. The solution was stirred quickly and aqueous ammonia having a concentration of 25% by weight in water was added dropwise until the pH of the solution reached 9. At this pH, a precipitate of Al₂O₃—Cr₂O₃.xH₂O solid was formed in the solution. The solution was left to stand for 24 hours, followed by suction filtering to obtain the precipitate. The precipitate in the solution was dried at 110° C. for 12 hours. The obtained Al₂O₃—Cr₂O₃.xH₂O solid was pulverized to smaller than 100 mesh (0.15 mm) and added to 2.4 mol/L H₂SO₄ solution. The amount of H₂SO₄ solution was 25 mL solution/g Al₂O₃—Cr₂O₃.xH₂O solid. The mixture of Al₂O₃—Cr₂O₃.xH₂O solid and the H₂SO₄ solution was stirred for 54.4 hours and filtered. After filtration, the resulting SO₄²⁻/Al₂O₃—Cr₂O₃.xH₂O was dried at 110° C. for 12 h, and calcinated at 500° C. for 6 hours to obtain pulverous SO₄²⁻/Al₂O₃—Cr₂O₃, a solid acid catalyst, which was weighed and recorded.

50 g of granular PET with a particle size of 1 mm, 10 g of SO₄²⁻/Al₂O₃—Cr₂O₃ catalyst and 100 g of deionized water were charged into a reactor. The reactor was sealed, stirred and heated to a constant temperature of 70° C. CO₂ was then charged, resulting in a reactor pressure of 17.1 MPa, and the reaction was carried out for 50 hours. After the reaction was completed, the pressure within the reactor was reduced to atmospheric pressure at a venting rate of 0.5 MPa/min. The products in the reactor were filtered and then the liquid phase was removed and subjected to distillation operation. After water was evaporated, pure ethylene glycol was obtained and then weighed to calculate the yield. The solid phase was dried, dissolved in a special organic solvent for 6 hours while stirring, and then filtered again. The liquid phase was removed for distillation. After the solvent was evaporated, pure terephthalic acid was obtained and weighed to calculate the yield. The solid phase was rinsed with deionized water to remove redundant solvent. After filtration, the residual solid was dried to constant weight to obtain SO₄²⁻/Al₂O₃—Cr₂O₃ catalyst and residual PET, which were weighed to obtain the degree of hydrolysis of PET.

### Example 8: Hydrolysis of PCT with SO₄²⁻/Al₂O₃—Cr₂O

30 g of Cr(NO₃)₃.9H₂O and 20 g of Al(NO₃)₃.9H₂O were dissolved in 1 L of distilled water. The solution was stirred quickly and aqueous ammonia having a concentration of 25% by weight in water was added dropwise until the pH of the solution reached 9. At this pH, a precipitate of Al₂O₃—Cr₂O₃.xH₂O solid was formed in the solution. The solution was left to stand for 24 hours, followed by suction filtering to obtain the precipitate. The precipitate in the solution was dried at 110° C. for 12 hours. The obtained Al₂O₃—Cr₂O₃.xH₂O solid was pulverized to smaller than 100 mesh (0.15 mm) and added to 4.3 mol/L H₂SO₄ solution. The amount of H₂SO₄ solution was 25 mL solution/g Al₂O₃—Cr₂O₃.xH₂O solid. The mixture of Al₂O₃—Cr₂O₃.xH₂O solid and the H₂SO₄ solution was stirred for 18 hours and filtered. After filtration, the resulting SO₄²⁻/Al₂O₃—Cr₂O₃.xH₂O was dried at 110° C. for 12 hours, and calcinated at 500° C. for 6 hours to obtain pulverous SO₄²⁻/Al₂O₃—Cr₂O₃, a solid acid catalyst, which was weighed and recorded.

Granular PCT with a length of 3 mm, a width of 3 mm and a height of 1.5 mm, 10 g of SO₄²⁻/Al₂O₃—Cr₂O₃ catalyst and 100 g of deionized water were charged into a reactor. The reactor was sealed, stirred and heated to a constant temperature of 270° C. CO₂ was then charged, resulting in a reactor pressure of 9.9 MPa, and the reaction was carried out for 30 hours. After the reaction was completed, the pressure within the reactor was reduced to atmospheric pressure at a venting rate of 0.5 MPa/min. The products in the reactor were filtered and then the liquid phase was removed and subjected to distillation operation. After water was evaporated, pure 1,4-cyclohexanedimethanol was obtained and then weighed to calculate the yield. The solid phase was dried, dissolved in a special organic solvent for 6 hours while stirring, and then filtered again. The liquid phase was removed for distillation. After the solvent was evaporated, pure terephthalic acid was obtained and weighed to calculate the yield. The solid phase was rinsed with deionized water to remove redundant solvent. After filtration, the residual solid was dried to constant weight to obtain SO₄²⁻/Al₂O₃—Cr₂O₃ catalyst and residual PCT, which were weighed to obtain the degree of hydrolysis of PCT.

### Example 9: Hydrolysis of PET with SO₄²⁻/Al₂O₃

50 g of Al(NO₃)₃.9H₂O was dissolved in 1 L of distilled water. The solution was stirred quickly and aqueous ammonia having a concentration of 25% by weight in water was added dropwise until the pH of the solution reached 9. At this pH, a precipitate of Al₂O₃.xH₂O solid was formed in the solution. The solution was left to stand for 24 hours, followed by suction filtering to obtain the precipitate. The precipitate in the solution was dried at 110° C. for 12 hours. The obtained Al₂O₃.xH₂O solid was pulverized to smaller than 100 mesh (0.15 mm) and added to 0.7 mol/L H₂SO₄ solution. The amount of H₂SO₄ solution was 25 mL solution/g Al₂O₃.xH₂O solid. The mixture of Al₂O₃.xH₂O solid and the H₂SO₄ solution was stirred for 58.8 hours and filtered. After filtration, the resulting SO₄²⁻/Al₂O₃.xH₂O was dried at 110° C. for 12 h, and calcinated at 500° C. for 6 hours to obtain pulverous SO₄²⁻/Al₂O₃, a solid acid catalyst, which was weighed and recorded.

50 g of block PET with a length of 10 mm, a width of 10 mm and a height of 3 mm, 10 g of SO₄²⁻/Al₂O₃ catalyst and 100 g of deionized water were charged into a reactor. The reactor was sealed, stirred and heated to a constant temperature of 330° C. CO₂ was then charged, resulting in a reactor pressure of 15.9 MPa, and the reaction was carried out for 2 hours. After the reaction was completed, the pressure within the reactor was reduced to atmospheric pressure at a venting rate of 0.5 MPa/min. The products in the reactor were filtered and then the liquid phase was removed and subjected to distillation operation. After water was evaporated, pure ethylene glycol was obtained and then weighed to calculate the yield. The solid phase was dried, dissolved in a special organic solvent for 6 hours while stirring, and then filtered again. The liquid phase was removed for distillation. After the solvent was evaporated, pure terephthalic acid was obtained and weighed to calculate the yield. The solid phase was rinsed with deionized water to remove redundant solvent. After filtration, the residual solid was dried to constant weight to obtain SO₄²⁻/Al₂O₃—Cr₂O₃ catalyst and residual PET, which were weighed to obtain the degree of hydrolysis of PET.

### Example 10: Hydrolysis of PEN with SO₄²⁻/Cr₂O₃

50 g of Cr(NO₃)₃.9H₂O was dissolved in 1 L of distilled water. The solution was stirred quickly and aqueous ammonia having a concentration of 25% by weight in water was added dropwise until the pH of the solution reached 9. At this pH, a precipitate of Cr₂O₃.xH₂O solid was formed in the solution. The solution was left to stand for 24 hours, followed by suction filtering to obtain the precipitate. The precipitate in the solution was dried at 110° C. for 12 hours. The obtained Cr₂O₃.xH₂O solid was pulverized to smaller than 100 mesh (0.15 mm) and added to 2.7 mol/L Cr₂(SO₄)₃ solution. The amount of H₂SO₄ solution was 25 mL solution/g Cr₂O₃.xH₂O solid. The mixture of Cr₂O₃.xH₂O solid and the H₂SO₄ solution was stirred for 23.6 hours and filtered. After filtration, the resulting SO₄²⁻/Cr₂O₃.xH₂O was dried at 110° C. for 12 hours, and calcinated at 500° C. for 6 hours to obtain pulverous SO₄²⁻/Cr₂O₃, a solid acid catalyst, which was weighed and recorded.

50 g of powder PEN with a particle size of 0.5 mm, 10 g of SO₄²⁻/Cr₂O₃ catalyst and 100 g of deionized water were charged into a reactor. The reactor was sealed, stirred and heated to a constant temperature of 270° C. CO₂ was then charged, resulting in a reactor pressure of 9.9 MPa, and the reaction was carried out for 30 hours. After the reaction was completed, the pressure within the reactor was reduced to atmospheric pressure at a venting rate of 0.5 MPa/min. The products in the reactor were filtered and then the liquid phase was removed and subjected to distillation operation. After water was evaporated, pure ethylene glycol was obtained and then weighed to calculate the yield. The solid phase was dried, dissolved in a special organic solvent for 6 hours while stirring, and then filtered again. The liquid phase was removed for distillation. After the solvent was evaporated, pure 2,6-naphthalic acid was obtained and weighed to calculate the yield. The solid phase was rinsed with deionized water to remove redundant solvent. After filtration, the residual solid was dried to constant weight to obtain SO₄²⁻/Cr₂O₃ catalyst and residual PEN, which were weighed to obtain the degree of hydrolysis of PEN.

### Example 11: Hydrolysis of PET with SO₄²⁻/ZrO₂—WO₃

50 g of ZrO(NO₃)₂.2H₂O was dissolved in 1 L of distilled water. The solution was stirred quickly and aqueous ammonia having a concentration of 25% by weight in water was added dropwise until the pH of the solution reached 9. At this pH, a precipitate of ZrO₂.xH₂O solid was formed in the solution. The solution was left to stand for 24 hours, followed by suction filtering to obtain the precipitate. The precipitate in the solution was dried at 110° C. for 12 hours. The obtained ZrO₂.xH₂O solid was pulverized to smaller than 100 mesh (0.15 mm). Na₂WO₄.2H₂O solution was heated and acidified by adding excessive hydrochloric acid into the solution to prepare tungstic acid H₂WO₄, which was dehydrated by heating at 110° C. to obtain WO₃. The WO₃ was added to 4.7 mol/L H₂SO₄ solution. The amount of H₂SO₄ solution was 25 mL solution/g WO₃ solid. The mixture of WO₃ solid, ZrO₂.xH₂O, and the H₂SO₄ solution was stirred for 63.2 hours and filtered. After filtration, the resulting SO₄²⁻/ZrO₂.xH₂O—WO₃.xH₂O was dried at 110° C. for 12 hours, and calcinated at 550° C. for 6 hours to obtain pulverous SO₄²⁻/ZrO₂—WO₃, an ultrastrong solid acid catalyst, which was weighed and recorded.

50 g of granular PET with a particle size of 1 mm, 10 g of SO₄²⁻/Al₂O₃ catalyst and 100 g of deionized water were charged into a reactor. The reactor was sealed, stirred and heated to a constant temperature of 190° C. CO₂ was then charged, resulting in a reactor pressure of 8.7 MPa, and the reaction was carried out for 0.1 hour. After the reaction was completed, the pressure within the reactor was reduced to atmospheric pressure at a venting rate of 0.5 MPa/min. The products in the reactor were filtered and then the liquid phase was removed and subjected to distillation operation. After water was evaporated, pure ethylene glycol was obtained and then weighed to calculate the yield. The solid phase was dried, dissolved in a special organic solvent for 6 hours while stirring, and then filtered again. The liquid phase was removed for distillation. After the solvent was evaporated, pure terephthalic acid was obtained and weighed to calculate the yield. The solid phase was rinsed with deionized water to remove redundant solvent. After filtration, the residual solid was dried to constant weight to obtain SO₄²⁻/ZrO₂—WO₃ catalyst and residual PET, which were weighed to obtain the degree of hydrolysis of PET.

### Example 12: Hydrolysis of PPA with SO₄²⁻/SiO₂—V₂O₅

30 g of Na₂SiO₃ and 20 g of V(NO₃)₅ were dissolved in 1 L of distilled water. The solution was stirred quickly and aqueous ammonia having a concentration of 25% by weight in water was added dropwise until the pH of the solution reached 9. At this pH, a precipitate of SiO₂—V₂O₅.xH₂O solid was formed in the solution. The solution was left to stand for 24 hours, followed by suction filtering to obtain the precipitate. The precipitate in the solution was dried at 110° C. for 12 hours. The obtained SiO₂—V₂O₅.xH₂O solid was pulverized to smaller than 100 mesh (0.15 mm) and added to 1 mol/L H₂SO₄ solution. The amount of H₂SO₄ solution was 25 mL solution/g SiO₂—V₂O₅.xH₂O solid. The mixture of SiO₂—V₂O₅.xH₂O solid and the H₂SO₄ solution was stirred for 28 hours and filtered. After filtration, the resulting SO₄²⁻/SiO₂—V₂O₅.xH₂O was dried at 110° C. for 50 hours, and calcinated at 550° C. for 6 hours to obtain pulverous SO₄²⁻/SiO₂—V₂O₅, an ultrastrong solid acid catalyst, which was weighed and recorded.

50 g of cuboid block PPA with a length of 6 mm, a width of 6 mm and a height of 2 mm, 10 g of SO₄²⁻/SiO₂—V₂O₅ catalyst and 100 g of deionized water were charged into a reactor. The reactor was sealed, stirred and heated to a constant temperature of 50° C. CO₂ was then charged, resulting in a reactor pressure of 21.9 MPa, and the reaction was carried out for 72 hours. After the reaction was completed, the pressure within the reactor was reduced to atmospheric pressure at a venting rate of 0.5 MPa/min. The products in the reactor were filtered and then the liquid phase was removed and subjected to liquid separation operation. After the water in the obtained aqueous phase was evaporated, pure propylene glycol was obtained and then weighed to calculate the yield. The obtained oil phase was PPA, which was weighed to obtain the degree of hydrolysis of PPA. The solid phase after filtration was hexanedioic acid and SO₄²⁻/SiO₂—V₂O₅ catalyst. Hexanedioic acid was dissolved in ethanol and filtered to recover the catalyst. Hexanedioic acid could be obtained after ethanol was evaporated.

### Example 13: Hydrolysis of PET with SO₄²⁻/TiO₂

50 g of Ti(SO₄)₂.8H₂O was dissolved in 1 L of distilled water. The solution was stirred quickly and aqueous ammonia having a concentration of 25% by weight in water was added dropwise until the pH of the solution reached 9. At this pH, a precipitate of TiO₂.xH₂O solid was formed in the solution. The solution was left to stand for 24 hours, followed by suction filtering to obtain the precipitate. The precipitate in the solution was dried at 110° C. for 12 hours. The obtained TiO₂.xH₂O solid was pulverized to smaller than 100 mesh (0.15 mm) and added to 3 mol/L H₂SO₄ solution. The amount of H₂SO₄ solution was 25 mL solution/g TiO₂.xH₂O solid. The mixture of TiO₂.xH₂O solid and the H₂SO₄ solution was stirred for 67.6 hours and filtered. After filtration, the resulting SO₄²⁻/TiO₂.xH₂O was dried at 110° C. for 12 h, and calcinated at 500° C. for 6 hours to obtain pulverous SO₄²⁻/TiO₂, an ultrastrong solid acid catalyst, which was weighed and recorded.

50 g of powder PET with a particle size of 1.0 mm, 10 g of SO₄²⁻/TiO₂ catalyst and 100 g of deionized water were charged into a reactor. The reactor was sealed, stirred and heated to a constant temperature of 250° C. CO₂ was then charged, resulting in a reactor pressure of 14.7 MPa, and the reaction was carried out for 50 hours. After the reaction was completed, the pressure within the reactor was reduced to atmospheric pressure at a venting rate of 0.5 MPa/min. The products in the reactor were filtered and then the liquid phase was removed and subjected to distillation operation. After water was evaporated, pure ethylene glycol was obtained and then weighed to calculate the yield. The solid phase was dried, dissolved in a special organic solvent for 6 hours while stirring, and then filtered again. The liquid phase was removed for distillation. After the solvent was evaporated, pure terephthalic acid was obtained and weighed to calculate the yield. The solid phase was rinsed with deionized water to remove redundant solvent. After filtration, the residual solid was dried to constant weight to obtain SO₄²⁻/TiO₂ catalyst and residual PET, which were weighed to obtain the degree of hydrolysis of PET.

### Example 14: Hydrolysis of PBT with SO₄²⁻/WO₃

50 g of Na₂WO₄.2H₂O solution was heated, and acidified by adding excessive hydrochloric acid into the solution to prepare tungstic acid H₂WO₄, which was dehydrated by heating at 110° C. to obtain WO₃. The WO₃ was added to 5 mol/L H₂SO₄ solution. The amount of H₂SO₄ solution was 25 mL solution/g WO₃ solid. The mixture of WO₃ solid and the H₂SO₄ solution was stirred for 32.4 hours and filtered. After filtration, the resulting SO₄²⁻/WO₃.xH₂O was dried at 110° C. for 12 hours and calcinated at 550° C. for 6 hours to obtain pulverous SO₄²⁻/WO₃, an ultrastrong solid acid catalyst, which was weighed and recorded.

50 g of granular PBT with a particle size of 1 mm, 10 g of SO₄²⁻/SnO₂ catalyst and 100 g of deionized water were charged into a reactor. The reactor was sealed, stirred and heated to a constant temperature of 110° C. CO₂ was then charged, resulting in a reactor pressure of 7.5 MPa, and the reaction was carried out for 24 hours. After the reaction was completed, the pressure within the reactor was reduced to atmospheric pressure at a venting rate of 0.5 MPa/min. The products in the reactor were filtered and then the liquid phase was removed and subjected to distillation operation. After water was evaporated, pure butanediol was obtained and then weighed to calculate the yield. The solid phase was dried, dissolved in a special organic solvent for 6 hours while stirring, and then filtered again. The liquid phase was removed for distillation. After the solvent was evaporated, pure terephthalic acid was obtained and weighed to calculate the yield. The solid phase was rinsed with deionized water to remove redundant solvent. After filtration, the residual solid was dried to constant weight to obtain SO₄²⁻/SnO₂ catalyst and residual PBT, which were weighed to obtain the degree of hydrolysis of PBT.

### Example 15: Hydrolysis of PBS with SO₄²⁻/ZrO₂—Al₂O₃—WO₃

50 g of Zr(SO₄)₂.4H₂O was dissolved in 1 L of distilled water. The solution was stirred quickly and aqueous ammonia having a concentration of 25% by weight in water was added dropwise until the pH of the solution reached 9. At this pH, a precipitate of ZrO₂.xH₂O solid was formed in the solution. The solution was left to stand for 24 hours, followed by suction filtering to obtain the precipitate. The precipitate in the solution was dried at 110° C. for 12 hours. The obtained ZrO₂.xH₂O solid was pulverized to smaller than 100 mesh (0.15 mm). 50 g of Al₂(SO₄)₃.18H₂O was dissolved in 1 L of distilled water. The solution was stirred quickly and aqueous ammonia having a concentration of 25% by weight in water was added dropwise until the pH of the solution reached 6.5. At this pH, a precipitate of Al₂O₃.xH₂O solid was formed in the solution. The solution was left to stand for 24 hours, followed by suction filtering to obtain the precipitate. The precipitate in the solution was dried at 110° C. for 12 hours. The obtained Al₂O₃.xH₂O solid was pulverized to smaller than 100 mesh (0.15 mm). Na₂WO₄.2H₂O solution was heated and acidified by adding excessive hydrochloric acid to prepare tungstic acid H₂WO₄ (slightly soluble in water), which was dehydrated by heating at 100° C. to obtain WO₃. The WO₃ was added to 1.4 mol/L H₂SO₄ solution. The amount of H₂SO₄ solution was 25 mL solution/g WO₃ solid. The mixture of ZrO₂.xH₂O solid, Al₂O₃.xH₂O solid, WO₃ solid and the H₂SO₄ solution was stirred for 72.0 hours and filtered. After filtration, the resulting SO₄²⁻/ZrO₂—Al₂O₃—WO₃.xH₂O was dried at 110° C. for 12 hours, and calcinated at 550° C. for 6 hours to obtain pulverous SO₄²⁻/ZrO₂—Al₂O₃—WO₃, an ultrastrong solid acid catalyst.

50 g of cuboid block PBS with a length of 8 mm, a width of 8 mm and a height of 3 mm, 10 g of SO₄²⁻/WO₃ catalyst and 100 g of deionized water were charged into a reactor. The reactor was sealed, stirred and heated to a constant temperature of 310° C. CO₂ was then charged, resulting in a reactor pressure of 20.7 MPa, and the reaction was carried out for 6.0 hours. After the reaction was completed, the pressure within the reactor was reduced to atmospheric pressure at a venting rate of 0.5 MPa/min. The products in the reactor were filtered and then the liquid phase was removed and subjected to fractional distillation. After water was evaporated, distillation was proceeded to obtain pure butanediol. The remnant portion was succinic acid which could be rinsed with ethanol and then filtered to obtain a pure product. The solid phase obtained in the first step of filtration was dried, dissolved in pure chloroform for 6 hours while stirring, and then filtered again. The liquid phase was removed for distillation. After chloroform was evaporated, pure PBS was obtained and weighed to calculate the yield. The solid phase was rinsed with ethanol to remove redundant chloroform solvent. After filtration, the residual solid was dried to constant weight to obtain SO₄²⁻/ZrO₂—Al₂O₃—WO₃ catalyst and residual PBS, which were weighed to obtain the degree of hydrolysis of PBS.

### Example 16: Hydrolysis of PET with SO₄²⁻/SiO₂—Al₂O₃

30 g of Na₂SiO₃ and 20 g of Al(NO₃)₃.9H₂O were dissolved in 1 L of distilled water. The solution was stirred quickly and aqueous ammonia having a concentration of 25% by weight in water was added dropwise until the pH of the solution reached 9. At this pH, a precipitate of SiO₂-Al₂O₃.xH₂O solid was formed in the solution. The solution was left to stand for 24 hours, followed by suction filtering to obtain the precipitate. The precipitate in the solution was dried at 110° C. for 12 hours. The obtained SiO₂—Al₂O₃.xH₂O solid was pulverized to smaller than 100 mesh (0.15 mm) and added to 3.4 mol/L H₂SO₄ solution. The amount of H₂SO₄ solution was 25 mL solution/g SiO₂—Al₂O₃.xH₂O solid. The mixture of SiO₂—Al₂O₃.xH₂O solid and the H₂SO₄ solution was stirred for 36.8 hours and filtered. After filtration, the resulting SO₄²⁻/SiO₂—Al₂O₃.xH₂O was dried at 110° C. for 50 hours, and calcinated at 550° C. for 6 hours to obtain pulverous SO₄²⁻/SiO₂—Al₂O₃, an ultrastrong solid acid catalyst, which was weighed and recorded.

50 g of powder PET with a particle size of 1 mm, 10 g of SO₄²⁻/SiO₂—Al₂O₃ catalyst and 100 g of deionized water were charged into a reactor. The reactor was sealed, stirred and heated to a constant temperature of 170° C. CO₂ was then charged, resulting in a reactor pressure of 13.5 MPa, and the reaction was carried out for 1 hour. After the reaction was completed, the pressure within the reactor was reduced to atmospheric pressure at a venting rate of 0.5 MPa/min. The products in the reactor were filtered and then the liquid phase was removed and subjected to distillation operation. After water was evaporated, pure ethylene glycol was obtained and then weighed to calculate the yield. The solid phase was dried, dissolved in a special organic solvent for 6 hours while stirring, and then filtered again. The liquid phase was removed for distillation. After the solvent was evaporated, pure terephthalic acid was obtained and weighed to calculate the yield. The solid phase was rinsed with deionized water to remove redundant solvent. After filtration, the residual solid was dried to constant weight to obtain SO₄²⁻/SiO₂—Al₂O₃ catalyst and residual PET, which were weighed to obtain the degree of hydrolysis of PET.

### Example 17: Hydrolysis of PET with NO₃/SiO₂—Al₂O₃

30 g of Na₂SiO₃ and 20 g of Al(NO₃)₃.9H₂O were dissolved in 1 L of distilled water. The solution was stirred quickly and aqueous ammonia having a concentration of 25% by weight in water was added dropwise until the pH of the solution reached 9. At this pH, a precipitate of SiO₂—Al₂O₃.xH₂O solid was formed in the solution. The solution was left to stand for 24 hours, followed by suction filtering to obtain the precipitate. The precipitate in the solution was dried at 110° C. for 12 hours. The obtained SiO₂—Al₂O₃.xH₂O solid was pulverized to smaller than 100 mesh (0.15 mm) and added to 3.4 mol/L HNO₃ solution. The amount of H₂SO₄ solution was 25 mL solution/g NO₃⁻/SiO₂—Al₂O₃.xH₂O solid. The mixture of NO₃SiO₂—Al₂O₃.xH₂O solid and the H₂SO₄ solution was stirred for 36.8 hours and filtered. After filtration, the resulting NO₃⁻/SiO₂-Al₂O₃.xH₂O was dried at 110° C. for 50 hours, and calcinated at 550° C. for 6 hours to obtain pulverous NO₃⁻/SiO₂—Al₂O₃, an ultrastrong solid acid catalyst, which was weighed and recorded.

50 g of powder PET with a particle size of 1 mm, 10 g of NO₃⁻/SiO₂—Al₂O₃ catalyst and 100 g of deionized water were charged into a reactor. The reactor was sealed, stirred and heated to a constant temperature of 170° C. CO₂ was then charged, resulting in a reactor pressure of 13.5 MPa, and the reaction was carried out for 1 hour. After the reaction was completed, the pressure within the reactor was reduced to atmospheric pressure at a venting rate of 0.5 MPa/min. The products in the reactor were filtered and then the liquid phase was removed and subjected to distillation operation. After water was evaporated, pure ethylene glycol was obtained and then weighed to calculate the yield. The solid phase was dried, dissolved in a special organic solvent for 6 hours while stirring, and then filtered again. The liquid phase was removed for distillation. After the solvent was evaporated, pure terephthalic acid was obtained and weighed to calculate the yield. The solid phase was rinsed with deionized water to remove redundant solvent. After filtration, the residual solid was dried to constant weight to obtain NO₃⁻/SiO₂—Al₂O₃ catalyst and residual PET, which were weighed to obtain the degree of hydrolysis of PET.

### Example 18: Hydrolysis of PET with PO₄³⁻/SiO₂—Al₂O₃

30 g of Na₂SiO₃ and 20 g of Al(NO₃)₃.9H₂O were dissolved in 1 L of distilled water. The solution was stirred quickly and aqueous ammonia having a concentration of 25% by weight in water was added dropwise until the pH of the solution reached 9. At this pH, a precipitate of SiO₂—Al₂O₃.xH₂O solid was formed in the solution. The solution was left to stand for 24 hours, followed by suction filtering to obtain the precipitate. The precipitate in the solution was dried at 110° C. for 12 hours. The obtained SiO₂—Al₂O₃.xH₂O solid was pulverized to smaller than 100 mesh (0.15 mm) and added to 3.4 mol/L H₃PO₄ solution. The amount of H₂SO₄ solution was 25 mL solution/g PO₄³⁻/SiO₂—Al₂O₃.xH₂O solid. The mixture of PO₄³⁻/SiO₂—Al₂O₃.xH₂O solid and the H₂SO₄ solution was stirred for 36.8 hours and filtered. After filtration, the resulting PO₄³⁻/SiO₂—Al₂O₃.xH₂O was dried at 110° C. for 50 hours, and calcinated at 550° C. for 6 hours to obtain pulverous PO₄³⁻/SiO₂—Al₂O₃, an ultrastrong solid acid catalyst, which was weighed and recorded.

50 g of powder PET with a particle size of 1 mm, 10 g of PO₄³⁻/SiO₂—Al₂O₃ catalyst and 100 g of deionized water were charged into a reactor. The reactor was sealed, stirred and heated to a constant temperature of 170° C. CO₂ was then charged, resulting in a reactor pressure of 13.5 MPa, and the reaction was carried out for 1 hour. After the reaction was completed, the pressure within the reactor was reduced to atmospheric pressure at a venting rate of 0.5 MPa/min. The products in the reactor were filtered and then the liquid phase was removed and subjected to distillation operation. After water was evaporated, pure ethylene glycol was obtained and then weighed to calculate the yield. The solid phase was dried, dissolved in a special organic solvent for 6 hours while stirring, and then filtered again. The liquid phase was removed for distillation. After the solvent was evaporated, pure terephthalic acid was obtained and weighed to calculate the yield. The solid phase was rinsed with deionized water to remove redundant solvent. After filtration, the residual solid was dried to constant weight to obtain PO₄³⁻/SiO₂—Al₂O₃ catalyst and residual PET, which were weighed to obtain the degree of hydrolysis of PET.

### Example 19: Hydrolysis of PET with TiO₂—Al₂O₃

30 g of Ti(SO₄)₂.8H₂O and 20 g of Al(NO₃)₃.9H₂O were dissolved in 1 L of distilled water. The solution was stirred quickly and aqueous ammonia having a concentration of 25% by weight in water was added dropwise until the pH of the solution reached 9. At this pH, a precipitate of TiO₂—Al₂O₃.xH₂O solid was formed in the solution. The solution was left to stand for 24 hours, followed by suction filtering to obtain the precipitate. The precipitate in the solution was dried at 110° C. for 12 hours. The obtained TiO₂—Al₂O₃.xH₂O solid was pulverized to smaller than 100 mesh (0.15 mm), dried at 110° C. for 12 h, and calcinated at 500° C. for 6 hours to obtain pulverous TiO₂—Al₂O₃, a solid acid catalyst, which was weighed and recorded.

50 g of powder PET with a particle size of 1 mm, 10 g of TiO₂—Al₂O₃ catalyst and 100 g of deionized water were charged into a reactor. The reactor was sealed, stirred and heated to a constant temperature of 290° C. CO₂ was then charged, resulting in a reactor pressure of 25.5 MPa, and the reaction was carried out for 18 hour. After the reaction was completed, the pressure within the reactor was reduced to atmospheric pressure at a venting rate of 0.5 MPa/min. The products in the reactor were filtered and then the liquid phase was removed and subjected to distillation operation. After water was evaporated, pure ethylene glycol was obtained and then weighed to calculate the yield. The solid phase was dried, dissolved in a special organic solvent for 6 hours while stirring, and then filtered again. The liquid phase was removed for distillation. After the solvent was evaporated, pure terephthalic acid was obtained and weighed to calculate the yield. The solid phase was rinsed with deionized water to remove redundant solvent. After filtration, the residual solid was dried to constant weight to obtain TiO₂—Al₂O₃ catalyst and residual PET, which were weighed to obtain the degree of hydrolysis of PET.

### Evaluation of Examples 1 to 19

The hydrolysis reactions in examples 1 to 19 were evaluated by measuring the degree of hydrolysis of the polyesters and the yields of dicarboxylic acids and polyols. Results showing the degree of hydrolysis and the yields for each of the examples are provided in Table 2.

TABLE 2
Results showing degree of hydrolysis and the yields of dicarboxylic
acids and polyols for each of the Examples 1 to 19

| | | | Degree of | Yield of | |
|---|---|---|---|---|---|
| | | | hydro- | dicar- | Yield of |
| | Poly- | | lysis, | boxylic | polyol, |
| No | ester | Catalyst | % | acid, % | % |
| 1 | PET | SO₄²⁻/V₂O₅ | 100.0 | 99.8 | 99.9 |
| 2 | PBT | SO₄²⁻/SnO₂ | 100.0 | 99.9 | 99.9 |
| 3 | PET | SO₄²⁻/TiO₂—Al₂O₃ | 84.2 | 83.9 | 84.1 |
| 4 | PBMA | SO₄²⁻/Al₂O₃—Cr₂O₃ | 46.8 | 45.9 | 46.1 |
| 5 | PET | SO₄²⁻/ZrO₂ | 13.2 | 13.1 | 13.0 |
| 6 | PBN | SO₄²⁻/CeO₂ | 100.0 | 99.9 | 99.8 |
| 7 | PET | SO₄²⁻/ZrO₂—Al₂O₃ | 100.0 | 99.8 | 99.8 |
| 8 | PCT | SO₄²⁻/Al₂O₃—Cr₂O₃ | 98.2 | 98.1 | 97.9 |
| 9 | PET | SO₄²⁻/Al₂O₃ | 67.8 | 66.9 | 67.1 |
| 10 | PEN | SO₄²⁻/Cr₂O₃ | 41.2 | 41.1 | 40.9 |
| 11 | PET | SO₄²⁻/ZrO₂—WO₃ | 6.8 | 6.2 | 6.3 |
| 12 | PPA | SO₄²⁻/SiO₂—V₂O₅ | 100.0 | 99.9 | 99.9 |
| 13 | PET | SO₄²⁻/TiO₂ | 100.0 | 99.8 | 99.9 |
| 14 | PBT | SO₄²⁻/WO₃ | 87.9 | 87.6 | 87.8 |
| 15 | PBS | SO₄²⁻/ZrO₂—Al₂O₃—WO₃ | 68.9 | 68.7 | 68.8 |
| 16 | PET | SO₄²⁻/SiO₂—Al₂O₃ | 21.2 | 21.1 | 21.0 |
| 17 | PET | NO₃⁻/SiO₂—Al₂O₃ | 17.6 | 17.5 | 17.6 |
| 18 | PET | PO₄³⁻/SiO₂—Al₂O₃ | 19.2 | 19.3 | 19.1 |
| 19 | PET | TiO₂—Al₂O₃ | 32.2 | 32.1 | 32.0 |

The Examples demonstrate the feasibility of hydrolyzing and degrading polyesters using solid acid catalysts. As the solid catalyst is recoverable and resuable, problems such as reactor corrosion and disposal of concentrated acids encountered in conventional methods can be avoided. Referring to Table 2, depending on the combination of polyester and catalyst used (for example, PET with SO₄²⁻/V₂O₅, PBT with SO₄²⁻/SnO₂, PBN with SO₄²⁻/CeO₂, PET with SO₄²⁻/ZrO—Al₂O₃, PPA with SO₄²⁻/SiO₂—V₂O₅, and PET with SO₄²⁻/TiO₂), the degree of hydrolysis was 100% showing complete degradation of the polyesters.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A method of hydrolyzing a polyester, the method comprising:
providing a first mixture comprising at least one polyester, and at least one solid acid catalyst; and
contacting the first mixture with carbon dioxide under conditions sufficient to hydrolyze the at least one polyester to form a second mixture comprising at least one dicarboxylic acid and at least one diol.

2. The method of claim 1, wherein providing a first mixture comprises providing at least one polyester is represented by Formula (I): 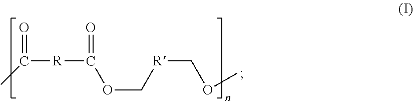
wherein
n is 100-500;
R is absent, C₁₋₈ alkylene, phenylene, naphthylene, or 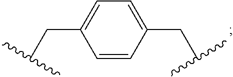 [SMILES: *Cc1ccc(C*)cc1]
and
R′ is absent, C₁₋₆ alkylene or C₃₋₈ cycloalkylene.

3. The method of claim 1, further comprising:
recovering the at least one dicarboxylic acid and the at least one diol from the second mixture.

4. (canceled)

5. The method of claim 3, wherein recovering the dicarboxylic acid and the diol from the second mixture comprises:
separating the second mixture into a first solid phase and a first liquid phase; and
separating the diol from the first liquid phase.

6.-7. (canceled)

8. The method of claim 3, wherein recovering the at least one dicarboxylic acid and the at least one diol from the second mixture comprises:
separating the second mixture into a first solid phase and a first liquid phase;
contacting the first solid phase with a solvent or an alkali to form a second solid phase and a second liquid phase;
converting a salt of the at least one dicarboxylic acid to the at least one dicarboxylic acid by contacting the second liquid phase with an acid; and
separating the at least one dicarboxylic acid from the second liquid phase.

9.-10. (canceled)

11. The method of claim 8, wherein contacting the first solid phase with an alkali comprises contacting with sodium hydroxide, potassium hydroxide, or both.

12. (canceled)

13. The method of claim 8, wherein contacting with an acid comprises contacting with an inorganic acid selected from hydrochloric acid, sulfuric acid, nitric acid, and phosphoric acid.

14. (canceled)

15. The method of claim 1, further comprising recovering the solid acid catalyst from the second mixture by:
separating the second mixture into a first solid phase and a first liquid phase;
contacting the first solid phase with a solvent or an alkali to form a second solid phase and a second liquid phase; and
washing the second solid phase to obtain the solid acid catalyst.

16.-17. (canceled)

18. The method of claim 15, wherein contacting the first solid phase with an alkali comprises contacting with sodium hydroxide, potassium hydroxide, or both, and contacting with a solvent comprises contacting with chloroform, ethanol, ethylether, DMF, DEF, and DMSO.

19.-21. (canceled)

22. The method of claim 1, wherein contacting the first mixture with the carbon dioxide occurs at a pressure of about 7.5 MPa to about 25.5 MPa and for about 0.1 hour to about 96 hours.

23. (canceled)

24. The method of claim 1, wherein forming the second mixture comprises forming the second mixture having at least one diol comprising ethylene glycol, propylene glycol, butanediol, hexanediol, cyclohexanedimethanol, heptanediol, octanediol, or any combination thereof.

25. The method of claim 1, wherein forming the second mixture comprises forming the second mixture where the at least one diol represented by:
R′(CH₂OH)₂,
wherein
R′ is C₃₋₈ cyclohexylene or —(CH₂)ₓ—, wherein x is 0, 1, 2, 3, or 4.

26. (canceled)

27. The method of claim 1, wherein forming the second mixture comprises forming the second mixture including at least one dicarboxylic acid comprising terephthalic acid, sebacic acid, p-naphthalic acid, 2,6-naphthalic acid, hexanedioic acid, succinic acid, propanedioic acid, azelaic acid, pimelic acid, suberic acid, glutaric acid, or any combination thereof.

28. The method of claim 1, wherein forming the second mixture comprise forming the second mixture having the at least one dicarboxylic acid comprising R(COOH)₂,
wherein
R is absent, ethylene, butylene, octylene, phenylene, or naphthylene, C₁₋₈ alkylene, phenylene, naphthylene, or

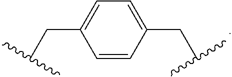 [SMILES: *Cc1ccc(C*)cc1]

29.-32. (canceled)

33. The method of claim 1, further comprising forming the solid acid catalyst by contacting at least one metal oxide with at least one strong acid, at least one salt of the strong acid, or both.

34.-35. (canceled)

36. The method of claim 33, wherein contacting at least one metal oxide comprises contacting with Fe₂O₃, Fe₃O₄, TiO₂, Al₂O₃, ZrO₂, V₂O₅, WO₃, Cr₂O₃, CeO₂, SnO₂, SiO₂—Al₂O₃, ZrO₂—WO₃, ZrO₂—Al₂O₃, TiO₂—Al₂O₃, ZrO₂—Al₂O₃—WO₃, SiO₂—V₂O₅, SiO₂—TiO₂, Al₂O₃—Cr₂O₃, or any combination thereof.

37. The method of claim 33, wherein contacting with at least one strong acid comprises contacting with SO₄²⁻, NO₃⁻, PO₄³⁻, or any combination thereof.

38.-41. (canceled)

42. A method of making a solid acid catalyst, the method comprising:
contacting at least one metal oxide with at least one strong acid ion to form a mixture comprising the solid acid catalyst.

43. (canceled)

44. The method of claim, further comprising:
isolating the solid catalyst from the mixture; and
drying the solid acid catalyst by heating to a first temperature of about 100° C. to about 150° C., and to a second temperature of about 400° C. to about 600° C.

45. The method of claim 44, wherein heating at the first temperature comprises heating for about 6 to about 50 hours and heating at the second temperature comprises heating for about 5 to about 7 hours.

46. (canceled)

47. The method of claim 42, wherein contacting with at least one strong acid ion includes contacting with the strong acid ion present in the mixture at a concentration of about 0.05 mol/L to about 5 mol/L.

48.-49. (canceled)

50. The method of claim 42, wherein contacting with the at least one metal oxide comprises contacting with Fe₂O₃, Fe₃O₄, TiO₂, Al₂O₃, ZrO₂, V₂O₅, WO₃, Cr₂O₃, CeO₂, SnO₂, SiO₂—Al₂O₃, ZrO₂—WO₃, ZrO₂—Al₂O₃, TiO₂—Al₂O₃, ZrO₂—Al₂O₃—WO₃, SiO₂—V₂O₅, SiO₂—TiO₂, Al₂O₃—Cr₂O₃, or any combination thereof.

51. (canceled)

52. The method of claim 42, wherein contacting with the strong acidic ion comprises contacting with SO₄²⁻, NO₃⁻, PO₄³⁻, or any combination thereof.

53.-54. (canceled)

55. A composition comprising:
at least one polyester and at least one solid acid catalyst, wherein the at least one solid acid catalyst is configured to hydrolyze the at least one polyester to the one or both of a dicarboxylic acid and a diol, the solid acid catalyst comprising at least one metal oxide and at least one strong acidic ion.

56. The composition of claim 55, further comprising at least one partially hydrolyzed polyester.

57. The composition of claim 55, wherein the dicarboxylic acid is selected from the group consisting of terephthalic acid, sebacic acid, p-naphthalic acid, 2,6-naphthalic acid, hexanedioic acid, succinic acid, propanedioic acid, azelaic acid, pimelic acid, suberic acid, and glutaric acid.

58. The composition of claim 55, wherein the dicarboxylic acid comprises:
R(COOH)₂,
wherein
R is absent, ethylene, butylene, octylene, phenylene, or naphthylene, C₁₋₈ alkylene, phenylene, naphthylene, or

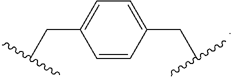 [SMILES: *Cc1ccc(C*)cc1]

59. The composition of claim 55, wherein the diol is selected from the group consisting of ethylene glycol, propylene glycol, butanediol, hexanediol, cyclohexanedimethanol, heptanediol, and octanediol.

60. The composition of claim 55, wherein the diol comprises:
R′(CH₂OH)₂,
wherein
R′ is C₃₋₈ cyclohexylene or —(CH₂)ₓ—, wherein x is 0, 1, 2, 3, or 4.

61. The composition of claim 55, wherein the at least one metal oxide is selected from the group consisting of Fe₂O₃, Fe₃O₄, TiO₂, Al₂O₃, ZrO₂, V₂O₅, WO₃, Cr₂O₃, CeO₂, SnO₂, SiO₂—Al₂O₃, ZrO₂—WO₃, ZrO₂—Al₂O₃, TiO₂—Al₂O₃, ZrO₂—Al₂O₃—WO₃, SiO₂—V₂O₅, SiO₂—TiO₂, and Al₂O₃—Cr₂O₃.

62. The composition of claim 55, wherein the at least one strong acidic ion is selected from the group consisting of SO₄²⁻, NO₃⁻, and PO₄³⁻.
